(19) United States
(12) Patent Application Publication
(10) Pub. No.: US 2010/0262081 A1
(43) Pub. Date: Oct. 14, 2010

(54) MICRONEEDLE ROLLER AND STAMP ADAPTED TO ENABLE THE REPLACEMENT OF MICRONEEDLES

(75) Inventors: Seung Seob Lee, Daejeon (KR); Boo Joon Sul, Daejeon (KR); Man Hee Han, Daejeon (KR)

Correspondence Address: Edwards Angell Palmer & Dodge LLP, P.O. Box 55874, Boston, MA, 02205, US

(73) Assignee: Miti Systems Inc., Daejeon (KR)

(21) Appl. No.: 12/789,527

(22) Filed: May 28, 2010

Related U.S. Application Data
(63) Continuation of application No. PCT/KR2009/001093, filed on Mar. 5, 2009.

(30) Foreign Application Priority Data
Aug. 7, 2008 (KR) 10-2008-0077360

(51) Int. Cl. A61M 5/00

(52) U.S. Cl. 604/173

(57) ABSTRACT

A microneedle-replaceable roller and stamp comprises a main body having a handle portion and a microneedle plate or sheet that is detachably mounted to the main body. According to the roller and stamp, inexpensive and sanitary application can be ensured.

### CROSS-REFERENCE TO RELATED APPLICATION

This is a continuation of International Application PCT/KR2009/001093, with an international filing date of Mar. 5, 2009, which claims the benefit of Korean Application No. 10-2008-0077360 filed Aug. 7, 2008, the entire contents of which are incorporated herein by reference.

### TECHNICAL FIELD

The present invention relates to a microneedle roller and stamp, and more particularly, to a microneedle roller and stamp having replaceable microneedles.

### BACKGROUND ART

In general, transdermal delivery of a cosmetic agent or a drug is disadvantageous in that since the stratum corneum makes it difficult for the agent and drug to penetrate therethrough, percutaneous or transcutaneous absorption of active ingredients thereof is extremely low. Particularly, if the active ingredients have a large molecular weight, the absorption becomes further low.

One proposed technique to solve this problem was to drug delivery method using microneedles. According to the technique, a fluid channel passing through a part or the whole of the epidermis is formed in a human skin by using microneedles, through which drug is delivered into the epidermal layer of the skin or a lower layer. According to this technique, even a drug having a large molecular weight such as insulin, hormone formulation and the like can be transdermally delivered easily. In addition, when a microneedle penetrates and stimulates the dermal layer, a burn or a scar can be naturally healed. Also, the skin is induced to produce collagen, so that the effect of improving skin tone and preventing skin aging can be maximized.

Currently, a microneedle roller is used to form such fluid channel. The microneedle roller has a structure in which a plurality of microneedles protrudes from the outer circumferential surface of a cylindrical roller head. When the roller head rotates in a state of being in close contact with the surface of a skin, the microneedles form a number of fluid channels in the skin while puncturing and penetrating the epidermis.

Such a conventional microneedle roller is configured to include a roller head formed integrally with microneedles and a main body, which makes it difficult to remove the microneedles from the roller head for replacement. Thus, replacement of the microneedles is not available when in use, which causes a problem of germ infection. In addition, if the microneedles are damaged or broken, it is required that the entire microneedle roller should be replaced with a new one.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in view of the above-mentioned problems occurring in the prior art, and it is an object of the present invention to provide a microneedle roller and a stamp that allow microneedles to be replaced inexpensively and sanitarily.

In order to accomplish the above object, in one aspect, the present invention provides a microneedle-replaceable roller, which comprises: a roller head including a cylindrical rotating body and a microneedle sheet mounted on the outer circumferential surface of the cylindrical rotating body, the microneedle sheet having a plurality of microneedles protrudingly formed thereon; and a main body including a support portion adapted to rotatably support the roller head and a handle portion extending from the support portion in a direction of going away from the roller head.

In another aspect, the present invention provides a microneedle-replaceable stamp, which comprises: a main body including a handle portion adapted to allow a user to grasp easily; and a microneedle plate including a plurality of microneedles formed protrudingly downwardly from the bottom surface thereof, the microneedle plate being detachably mounted to the main body.

According to the microneedle rollers and stamps, when the microneedles are damaged or contaminated, they can be replaced easily and sanitarily, which provides user convenience.

The above features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIG. 1 is an assembled perspective view illustrating a microneedle-replaceable roller according to a first embodiment of the present invention;

FIG. 2 is an exploded perspective view illustrating the roller of FIG. 1;

FIG. 3 is a perspective view illustrating a cylindrical rotating body of the roller head of the roller of FIG. 1;

FIG. 4 is a top perspective view illustrating a microneedle sheet of the roller head of the roller of FIG. 1;

FIG. 5 is an exploded perspective view illustrating a microneedle-replaceable roller according to a second embodiment of the present invention;

FIG. 6 is an exploded perspective view illustrating a microneedle-replaceable roller according to a third embodiment of the present invention;

FIG. 7 is a perspective view illustrating a cylindrical rotating body of the roller head of the roller of FIG. 6;

FIG. 8 is a bottom perspective view illustrating a microneedle sheet of the roller head of the roller of FIG. 6;

FIG. 9 is a side view illustrating a microneedle-replaceable stamp according to a first embodiment of the present invention;

FIG. 10 is a partial sectional perspective view illustrating a microneedle-replaceable stamp according to a second embodiment of the present invention;

FIG. 11 is an exploded perspective view illustrating the stamp of FIG. 10;

FIG. 12 is a perspective view illustrating the microneedle plate of the stamp of FIG. 10;

FIG. 13 is another perspective view illustrating the stamp of FIGS. 10; and

FIG. 14 is an exploded perspective view illustrating a microneedle-replaceable stamp according to a third embodiment of the present invention.

### DETAILED DESCRIPTION OF THE DISCLOSURE

The present invention will be hereinafter described in detail in connection with the preferred embodiments with reference to the accompanying drawings. However, these embodiments of the present invention are merely illustrative of easy explanation on the contents and scope of the technical spirit of the present invention, but the technical scope of the present invention is not limited or modified thereby. Also, it will be understood by those skilled in the art that various modifications and variations can be made to the present invention without departing from the spirit and scope of the appended claims based on the illustrative embodiments.

Now, the preferred embodiments of a microneedle-replaceable roller according to the present invention will be described in detail with reference to the accompanying drawings.

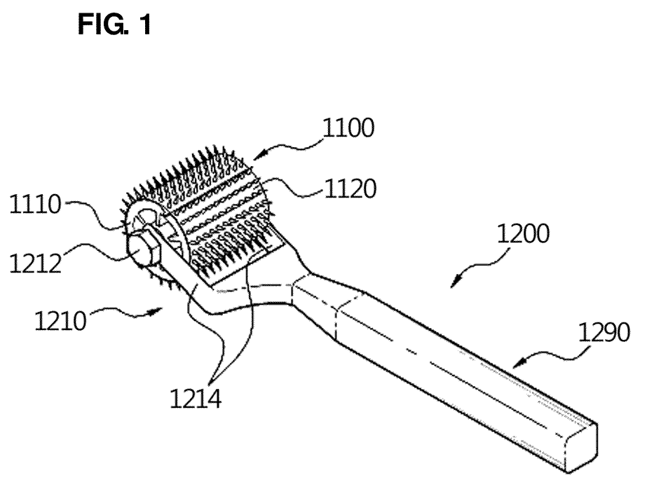

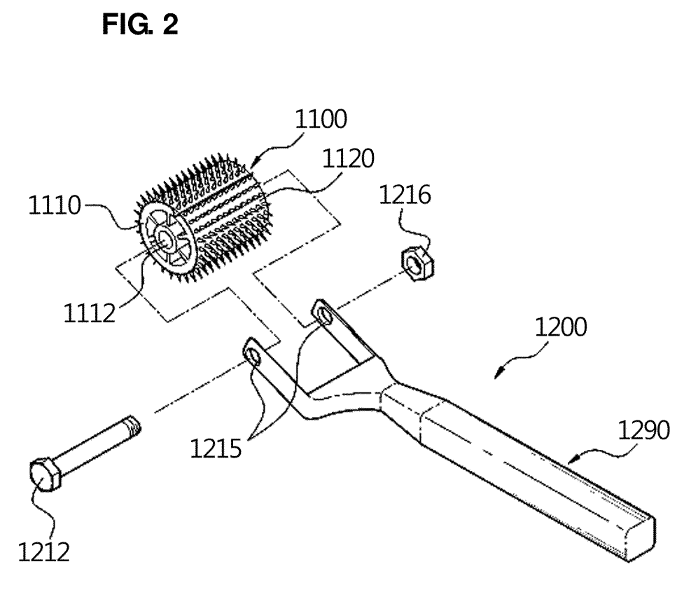

FIG. 1 is an assembled perspective view illustrating a microneedle-replaceable roller according to a first embodiment of the present invention, and FIG. 2 is an exploded perspective view illustrating the microneedle-replaceable roller of FIG. 1.

Referring to FIGS. 1 and 2, the microneedle-replaceable roller according to the first embodiment of the present invention includes a roller head 1100 and a main body 1200.

The roller head 1100 includes a cylindrical rotating body 1110 and a microneedle sheet 1120.

The cylindrical rotating body 1110 has a central hollow portion 1112 formed centrally in the cylindrical rotating body in the direction of a rotational axis thereof, and a microneedle sheet 1120 is wrappingly mounted on the outer circumferential surface of the cylindrical rotating body.

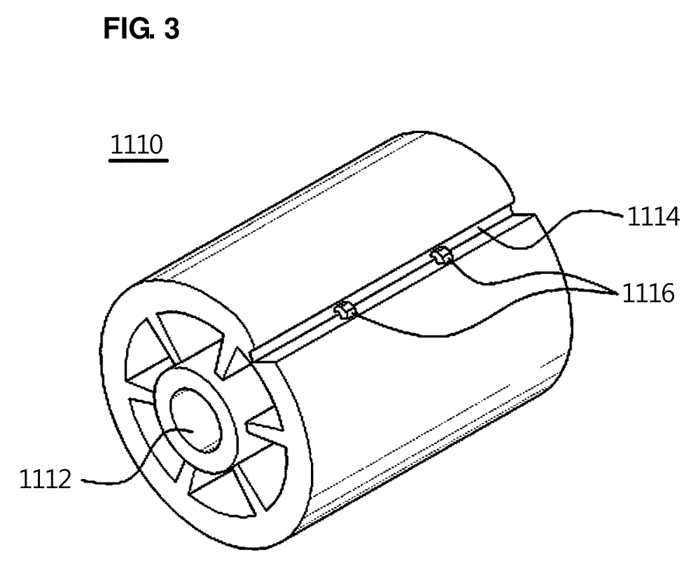

FIG. 3 is a perspective view illustrating a cylindrical rotating body of a roller head of the microneedle roller adapted to enable the replacement of microneedles shown in FIG. 1.

Referring to FIG. 3, the cylindrical rotating body 1110 includes a retaining groove 1114 formed on the outer circumferential surface thereof in a transverse direction thereof. The retaining groove 1114 may be formed in diverse shapes, but in this embodiment, it is formed in such a fashion that an upper portion thereof is larger in width than a lower portion thereof so as to allow a hook-shaped projection 1126 of the microneedle sheet 1120 to be hookingly retained therein.

The retaining groove 1114 includes a pair of retaining protrusions 1116 formed on the inner surface thereof. Each of the retaining protrusions 1116 is bent perpendicularly inwardly from the inner surface of the retaining groove 1114.

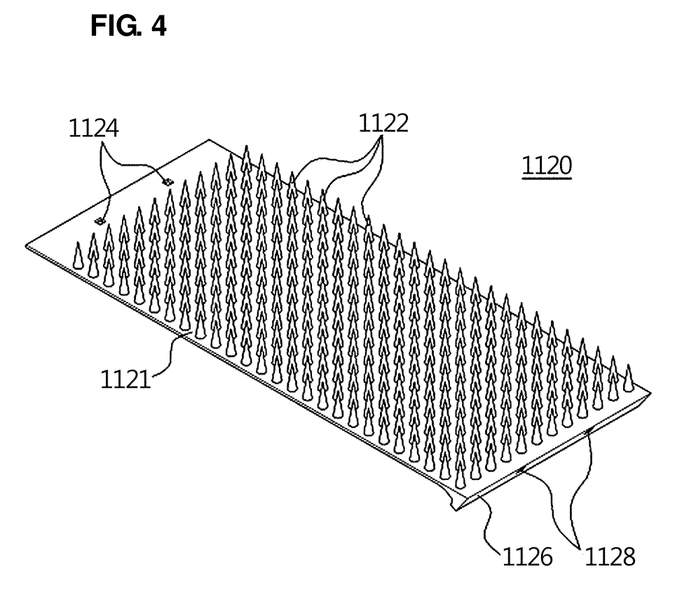

FIG. 4 is a top perspective view illustrating a microneedle sheet of the roller head of the microneedle-replaceable roller of FIG. 1.

Referring to FIG. 4, the microneedle sheet 1120 includes a sheet body 1121, a plurality of microneedles 1122, a pair of sheet openings 1124 and the projection 1126. The plurality of microneedles 1122 is formed protrudingly on the top surface of the microneedle sheet 1120 in such a fashion as to be spaced apart from each other at regular intervals. The protruding height of each microneedle may vary depending on the use purpose. For example, in case of forming a fluid channel through which the microneedles penetrate the epidermal layer to reach the dermal layer, the protruding height of the microneedle ranges from about 200 μm to 750 μm. The microneedles may be arranged at regular intervals to form a plurality of rows, and are preferably arranged more densely. The reason for this is because a number of channels can be formed at a skin site having the same area.

The sheet body 1121 of the microneedle sheet includes a pair of square sheet openings 1124 formed at one end thereof so as to allow the retaining protrusions 1116 formed on the retaining groove 1114 to be insertedly fitted thereto, and the hook-shaped retaining projection 1126 formed protrudingly at the other end thereof so as to be hookingly retained in the retaining groove 1114. The retaining projection 1126 protrudes downwardly from the bottom surface opposite to the top surface of the sheet body where the microneedles 1122 are formed. The retaining projection 1126 includes a pair of indents 1128 formed thereon so as to accommodate the retaining protrusions 1116 therein.

The sheet body 1121 is preferably flexible and has elasticity. If the sheet body 1121 has elasticity, the microneedle sheet 1120 can be brought into close contact with the outer circumferential surface of the cylindrical rotating body 1110.

The microneedle sheet 1120 can be engaged with the cylindrical rotating body 1110 in the following manner.

First, the sheet openings 1124 of the microneedle sheet 1120 are retainingly fitted on the retaining protrusions 1116. Then, the microneedle sheet 1120 is wrapped around the outer circumferential surface of the cylindrical rotating body 1110 and the retaining projection 1126 is hookingly retained in the retaining groove 1114 formed on the outer circumferential surface of the cylindrical rotating body 1110. The retaining protrusions 1116 are bent perpendicularly toward the sheet openings 1124, so that once the microneedle sheet 1120 is engaged with the cylindrical rotating body 1110, it may not be easily separated from the cylindrical rotating body 1110. The retaining protrusions 1116 which have penetratingly passed through the sheet openings 1124 are accommodated in the indents 1128 formed on a side of the retaining projection 1126. The microneedle sheet 1120 can be securely engaged with the cylindrical rotating body 1110 in this manner, and it can be disengaged from the cylindrical rotating body 1110 in the reverse order.

The retaining protrusions 1116 preferably have elasticity. The reason for this is because it is easy for the microneedle sheet 1120 to be retainingly fitted on the retaining protrusions 1116, and an tensile force is applied to the microneedle sheet 1120 after the microneedle sheet 1120 is securely engaged with the cylindrical rotating body 1110, so that the microneedle sheet 1120 may not be easily separated from the cylindrical rotating body 1110.

The main body 1200 includes a support portion 1210 and a handle portion 1290.

The support portion 1210 may have various structures so as to be able to rotatably support the roller head 1100 and be coupled with the handle portion 1290. Referring to FIGS. 1 and 2, for example, the support portion 1210 includes an engagement rod 1212 adapted to penetratingly pass through a hollow portion 1112 formed centrally in the cylindrical rotating body 1110 in the direction of a rotational axis of the cylindrical rotating body 1110, a pair of clamp arms 1214 including one end portion thereof having through-holes 1215 through which the engagement rod 1212 can pass and the other end portion integrally extending to the handle portion 1290, and a nut 1216 that is to be engaged with a distal end of the engagement rod 1212.

The engagement rod 1212 has threads formed on the outer circumferential surface of the distal end thereof so as to allow the nut 1216 to be screw-engaged therewith. It will be apparent to those skilled in the art that although the screw engagement of the nut 1216 with the engagement rod 1212 has been described in this embodiment, other engagement methods including, e.g., a pin engagement may also be employed.

Accordingly, the roller head 1100 can be assembled with the main body 1200 by positioning the roller head 1100 between the pair of clamp arms 1214 and the engagement rod 1212 passes through the through-holes 1215 of the clamp arms 1214 and the central hollow portion 1112 of the roller head 1100, and screw-engaging the nut 1216 with the threaded portion of the engagement rod 1212, and it can be disassembled from the main body 1200 in the reverse order.

Although the assembly of the roller head 1100 with the main body 1200 using the one engagement rod 1212 passing through the hollow portion and the nut 1216 has been described, the present invention should not be limited thereto. For example, threads may be formed on the inner circumferential surface of the hollow portion and two bolts may be engaged with the threads so as to rotatably fix the roller head instead of using the engagement rod.

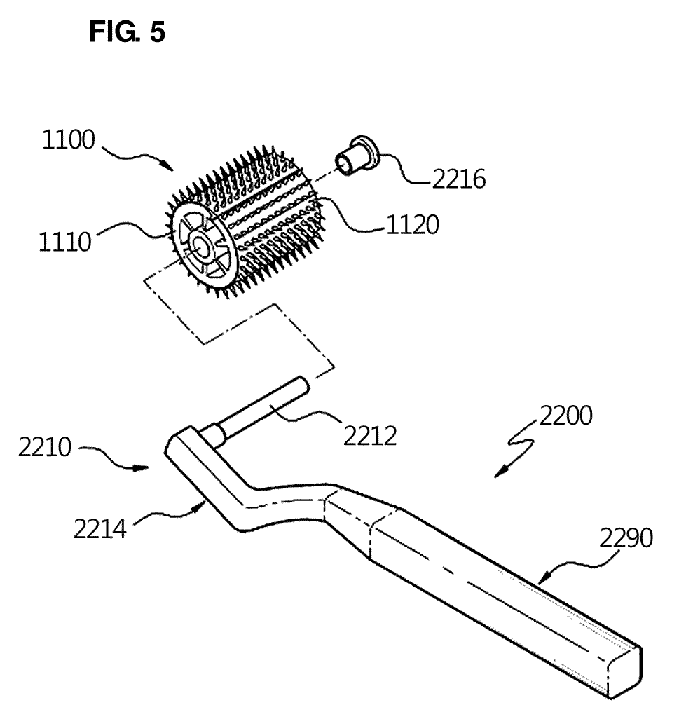

FIG. 5 is an exploded perspective view illustrating a microneedle-replaceable roller according to a second embodiment of the present invention.

Since the microneedle-replaceable roller of the second embodiment has the same structure as that of the first embodiment except for its support portion 2210, description of the support portion 2210 will be given while description of the other elements of the structure is omitted.

The support portion 2210 according to this embodiment includes a clamp arm 2214 having an engagement rod 2212 adapted to penetratingly pass through the hollow portion 1112 formed centrally in the cylindrical rotating body 1110 in the direction of the rotational axis of the cylindrical rotating body 1110, and a fastening member 2216 which is to be press-fittingly engaged with a distal end of the engagement rod 2212 such that the roller head 1100 is interposed between the clamp arm 2214 and the fastening member 2216.

It will be apparent to those skilled in the art that although the fitting engagement of the fastening member 2216 with the engagement rod 2212 has been described in this embodiment, the screw engagement therebetween may also be employed. In addition, the clamp arm may take another type of structure in which it is engaged with both the engagement rod 2212 and the handle portion 2290.

Besides, although it has been described that the clamp arm 2214 has the engagement rod 2212 adapted to penetratingly pass through the hollow portion 1112, the clamp arm 2214 may be constructed in such a manner that the clamp arm 2214 has a short engagement rod which does not entirely penetratingly pass through the hollow portion 1112 and another clamp arm is provided which has a short engagement rod engaging with both the engagement rod 2212 and the handle portion 2290.

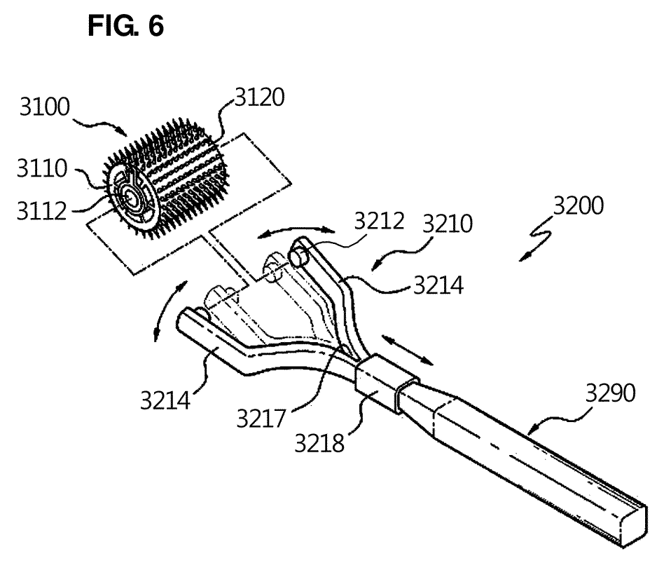

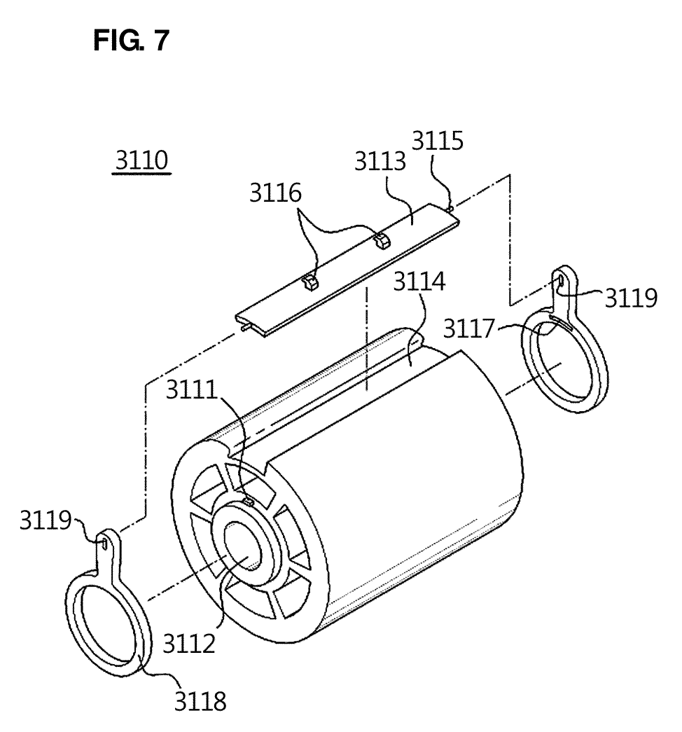

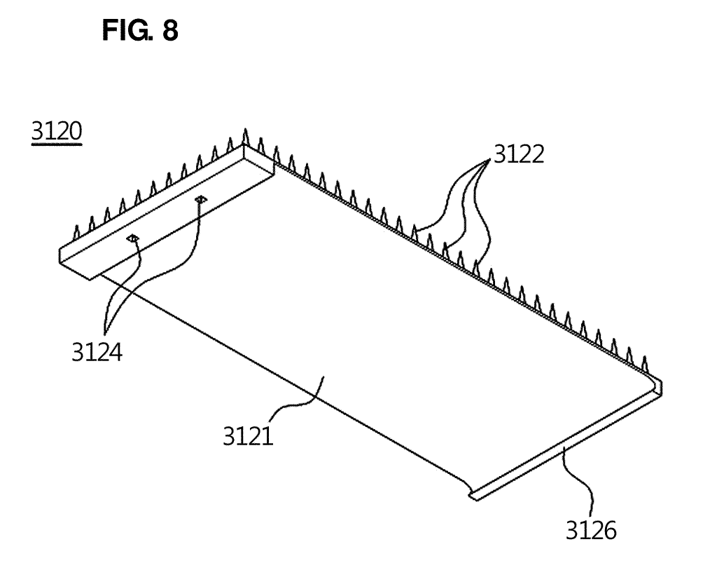

FIG. 6 is an exploded perspective view illustrating a microneedle-replaceable roller according to a third embodiment of the present invention, FIG. 7 is a perspective view illustrating a cylindrical rotating body of the roller head of the roller of FIG. 6, and FIG. 8 is a bottom perspective view illustrating a microneedle sheet of the roller of FIG. 6.

Referring to FIG. 6, the roller according to the third embodiment includes a roller head 3100 and a main body 3200.

The roller head 3100 includes a cylindrical rotating body 3110 and a microneedle sheet 3120.

The cylindrical rotating body 3110 has a central hollow portion 3112 formed centrally in the cylindrical rotating body in the direction of a rotational axis thereof, and a microneedle sheet 3120 is wrappingly mounted on the outer circumferential surface of the cylindrical rotating body.

The cylindrical rotating body 3110 has a retaining groove 3114 formed on the outer circumferential surface thereof in a transverse direction perpendicular to a circumferential direction thereof, as shown in FIG. 7. A retaining bar 3113 is seated in the retaining groove 3114, and side covers 3118 are joined to sides of the cylindrical rotating body 3110.

Unlike the cylindrical rotating body 1110 described by referring to FIG. 3, the cylindrical rotating body 3110 includes two retaining protrusions 3116 formed on the top surface of the retaining bar 3113, and the retaining bar 3113 is securely fixed to the cylindrical rotating body 3110 by means of the side covers 3118 joined to the sides of the cylindrical rotating body 3110.

The retaining bar 3113 includes a lug 3115 formed at each of both side ends thereof so as to be engaged with each of the side covers 3118. Each of the side covers 3118 has an engagement hole 3119 formed at an upper portion thereof so as to allow the each lug 3115 of the retaining bar 3113 to be fittingly inserted thereto, and an elongated fixing groove 3117 formed partially on an inner circumferential edge thereof so as to allow a protrusion 3111 formed at each of both side ends of the cylindrical rotating body 3110 to be fittingly fixed thereto.

The retaining bar 3113 is positioned within the retaining groove 3114 of the cylindrical rotating body 3110, and then the two opposed lugs 3115 of the retaining bar 3113 and the two opposed protrusions 3111 of the cylindrical rotating body 3110 are, respectively, fitted into the engagement holes 3119 and the fixing groove 3117 of the side covers 3118, resulting that the side covers 3118 support the retaining bar 3113 while being fixedly joined to the cylindrical rotating body 3110.

Referring to FIG. 8, the microneedle sheet 3120 includes a sheet body 3121, a plurality of microneedles 3122, a pair of sheet openings 3124 and a projection 3126.

The sheet body 3121 includes the microneedles 3122 formed on one surface thereof and a protruded portion protruding at one end of the other surface thereof. The protruded portion has a pair of sheet openings 3124 so as to allow the retaining protrusions 3116 formed on the retaining bar 3113 to be insertedly fitted thereto. Also, the sheet body 3121 includes a hook-shaped retaining projection 3126 formed downwardly protrudingly at the other end thereof so as to be hookingly retained in the retaining groove 3114.

The microneedle sheet 3120 can be engaged with the cylindrical rotating body 3110 in the following manner.

The retaining bar 3113 is securely fixed to the cylindrical rotating body 3110 using the side covers 3118, the sheet openings 3124 of the microneedle sheet 3120 are then retainingly fitted on the retaining protrusions 3116, and the microneedle sheet 3120 is then wrapped around the outer circumferential surface of the cylindrical rotating body 3110. Thereafter, the hook-shaped retaining protrusion 3126 is hookingly retained in the retaining groove 3114 formed on the outer circumferential surface of the cylindrical rotating body 3110. In this manner, the microneedle sheet 3120 can be securely engaged with the cylindrical rotating body 3110, and can be disengaged from the cylindrical rotating body 3110 in the reverse order.

Since the main body 3200 of the roller of this embodiment has the same structure as that of the first embodiment described by referring to FIGS. 1 and 2 except for its support portion 3210, the support portion 3210 only will be described hereinafter.

The support portion 3210 according to this embodiment includes a pair of clamp arms 3214 each having one free end at or near the roller head 3100 and the other end fixed to the handle portion 3290. The support portion 3210 is constructed such that the width between the clamp arms 3214 is gradually increased toward the free ends of the clamp arms 3214. A width adjustment ring 3218 is provided to surround a portion 3217 at which the fixed ends of the clamp arms are branched off. In this case, the fixed ends can be branched off at or near the position where the support portion 3210 and the handle portion 3290 are joined. For example, the fixed ends can be branched off directly from the handle portion 3290. Alternatively, they can integrally extend from the handle portion 3290 in a predetermined distance and then be branched off. An elastic force acts on the pair of camp arms 3214 in a direction of going away from each other.

The width adjustment ring 3218 is aimed at adjusting the width between the clamp arms 3214. When the width adjustment ring 3218 is forcedly pushed toward the roller head 3100, the width between the clamp arms 3214 becomes smaller. In this case, two opposed engagement rods 3212 formed on the inner sides of the free ends of the clamp arms 3214 become fitted into the hollow portion 3112 thereby rotatably supporting the roller head 3100.

On the contrary, when the width adjustment ring 3218 is forcedly pushed toward the handle portion 3290, the width between the clamp arms 3214 becomes larger so that the roller head 3100 can be detached from the main body 3200.

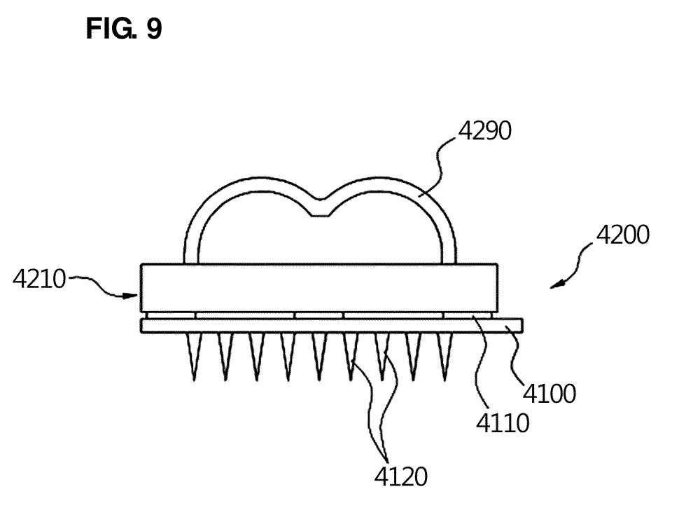

FIG. 9 is a side view illustrating a microneedle-replaceable stamp according to a first embodiment of the present invention.

Referring to FIG. 9, the microneedle-replaceable stamp includes a main body 4200 and a microneedle plate 4100.

The main body 4200 includes a handle portion 4290 adapted to allow a user to grasp easily and a flat microneedle-attaching portion 4210 disposed beneath the bottom of the handle portion 4290.

The microneedle plate 4100 includes a plurality of microneedles 4120 formed protrudingly downwardly from the bottom surface thereof and an adhesive layer 4110 coated on the top surface thereof so as to be detachably mounted on the bottom of the microneedle-attaching portion 4210 of the main body 4200.

If the microneedles are damaged or contaminated, the microneedle plate 4100 is removed and a new microneedle plate can be attached on the microneedle-attaching portion 4210 of the main body 4200. A protective film may be attached on the microneedle plate so as to protect the adhesive layer, and is removed and attached when in use.

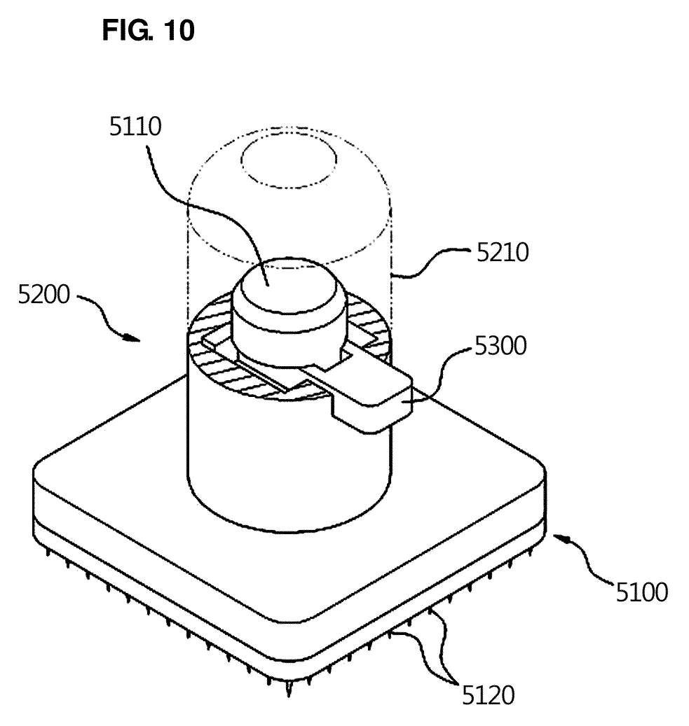

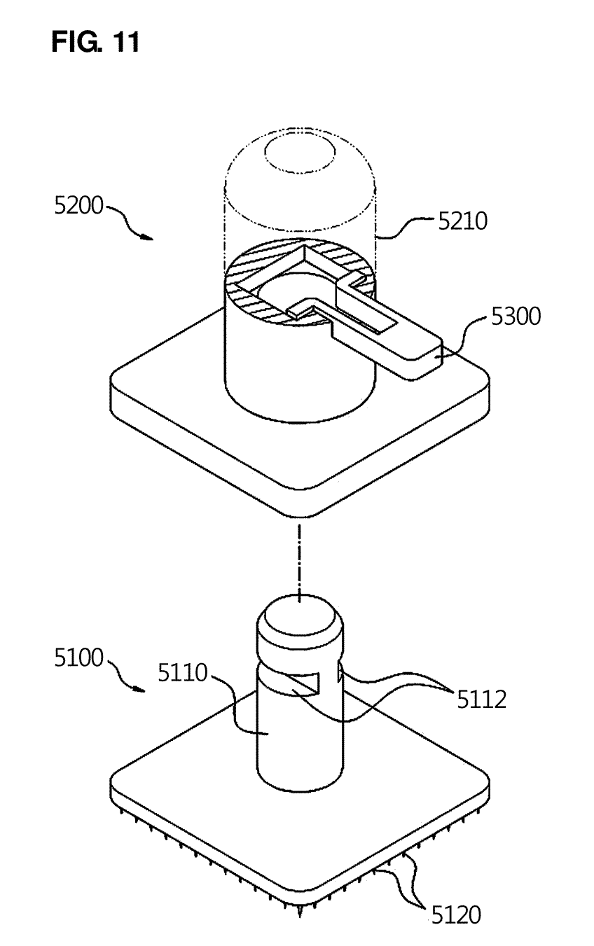

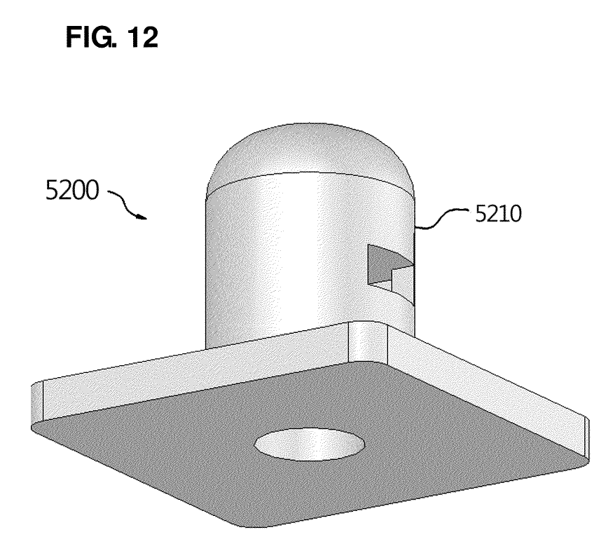

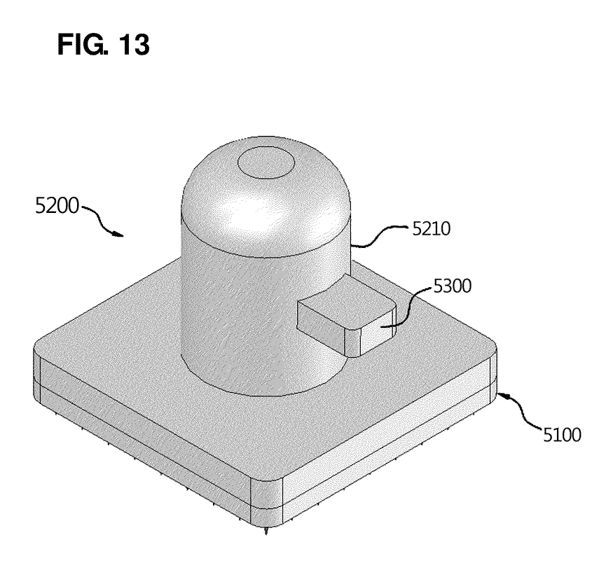

FIGS. 10 to 13 illustrate a microneedle-replaceable stamp according to a second embodiment of the present invention.

Referring to FIGS. 10 to 13, the microneedle-replaceable stamp according to the second embodiment of the present invention includes a main body 5200, a microneedle plate 5100, and a retaining member 5300.

The main body 5200 includes a cylindrical accommodating portion 5210 formed vertically protrudingly on the top surface of the main body 5200. The accommodating portion 5210 may be used a handle. Although it is described that the accommodating portion and others herein are cylindrical, the cylindrical shape is a non-limiting example and it thus can be another shape so long as the function of the portions does not change.

The microneedle plate 5100 includes a cylindrical fitting portion 5110 formed on the top surface thereof so as to be fitted into the accommodating portion 5210. The cylindrical fitting portion 5110 has one or more guide slots 5112 formed along the circumference thereof.

The retaining member 5300 is horizontally mounted in the accommodating portion 5210 of the main body in such a fashion as to pass through the accommodating portion 5210. The retaining member 5300 is movable inside the accommodating portion 5210 of the main body along the guide slot(s) 5112 formed on the cylindrical fitting portion 5110, so that the microneedle plate 5100 can be securely engaged with the main body 5200.

The engagement between the microneedle plate 5100 and the main body 5200 will be described hereinafter in more detail.

The cylindrical fitting portion 5110 of the microneedle plate is insertedly fitted into the accommodating portion 5210 of the main body 5200. Then, when the retaining member 5300 is forcedly pushed toward the accommodating portion 5210, it advances along the guide slot(s) 5112 formed on the cylindrical fitting portion 5110 so as to allow the microneedle plate 5100 to be securely engaged with the main body 5200. On the other hand, when the retaining member 5300 is forcedly pulled outwardly from the accommodating portion 5210, it retracts along the guide slot(s) 5112 so as to allow the microneedle plate 5100 to be disengaged from the main body 5200.

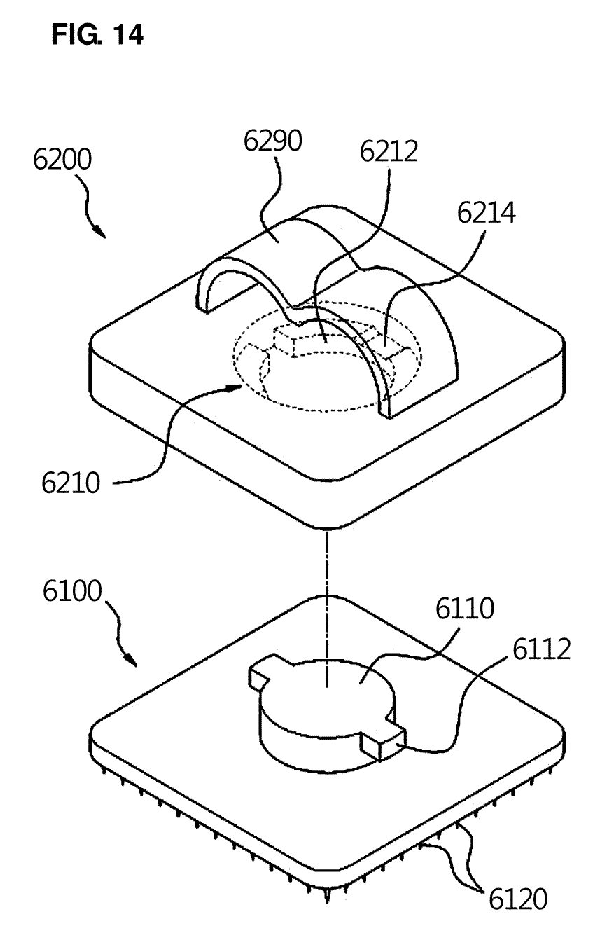

FIG. 14 is an exploded perspective view illustrating a microneedle-replaceable stamp according to a third embodiment of the present invention.

Referring to FIG. 14, the microneedle-replaceable stamp according to the third embodiment of the present invention includes a main body 6200 and a microneedle plate 6100.

The microneedle plate 6100 includes a cylindrical fitting portion 6110 formed on the top surface thereof and having a pair of opposed fixing protrusions 6112.

The main body 6200 includes a recessed portion 6210 formed therein so as to allow the fitting portion 6110 to be fitted thereto. The recessed portion 6210 includes a rotary guide portion 6212 adapted to support and guide the opposed fixing protrusions 6112 of the fitting portion 6110 when the microneedle plate 6100 rotates relative to the main body 6200. The rotary guide portion 6212 includes a fixing groove 6214 formed on the circumferential end face thereof so that when the fitting portion 6110 of microneedle plate 6100 rotates by a predetermined angle in a state of being fitted into the recessed portion 6210 of the main body 6200, the opposed fixing protrusions 6112 of the fitting portion 6110 can be retained in the fixing groove 6241 so as to cause the microneedle plate 6100 to be securely engaged with the main body 6200.

The engagement between the microneedle plate 6100 and the main body 6200 will be described hereinafter in more detail.

When the microneedle plate 6100 is rotated in a state where the fitting portion 6110 of the microneedle plate 6100 is fitted into the recessed portion 6210 of the main body 6200, the opposed fixing protrusions 6112 formed on the fitting portion 6110 of the microneedle plate 6100 rotate along the rotary guide portion 6212 formed on the recessed portion 6210 of the main body 6200. When the fitting portion 6110 rotates by a predetermined angle, the opposed fixing protrusions 6112 of the fitting portion 6110 are retained in the fixing grooves 6214 formed on the rotary guide portion 6212 so that the microneedle plate 6100 is securely engaged with the main body 6200. On the other hand, when the microneedle plate 6100 rotates in the opposite direction to the rotation direction by the same angle as that in the engagement operation relative to the main body 6200, it is disengaged from the main body.

While the microneedle-replaceable rollers and stamps according to the present invention have been described with reference to the illustrative embodiments, it is not to be restricted by the embodiments but only by the appended claims. It is to be appreciated that those skilled in the art can variously change or modify the embodiments without departing from the scope and spirit of the present invention. Therefore, this modification and change of the embodiments will fall within the protection scope of the present invention as long as it is apparently understood by those skilled in the art.

For example, a microneedle-replaceable roller may be constructed by combination of the main body according to the first embodiment and the roller head according to the third embodiment, or vice versa.

In addition, although it has been described that the microneedle sheet is securely engaged with the cylindrical rotating body using the sheet openings and the retaining projection formed on the microneedle sheet, the microneedle sheet may also be securely attached to the cylindrical rotating body using an adhesive layer formed on the microneedle sheet.

According to the present invention, it is possible to provide a microneedle roller or stamp that allows the microneedles thereof to be replaced inexpensively and sanitarily.

What is claimed is:

1. A microneedle-replaceable roller, comprising:
a roller head including a cylindrical rotating body and a microneedle sheet wrappingly mounted on the outer circumferential surface of the cylindrical rotating body, the microneedle sheet having a plurality of microneedles protrudingly formed thereon; and
a main body including a support portion adapted to rotatably support the roller head and a handle portion extending from the support portion in a direction of going away from the roller head.

2. The microneedle-replaceable roller according to claim 1, wherein the support portion comprises:
an engagement rod adapted to penetratingly pass through a hollow portion formed centrally in the cylindrical rotating body in the direction of a rotational axis of the cylindrical rotating body; and
a pair of clamp arms fixedly coupled to both ends of the engagement rod and integrally extending to the handle portion.

3. The microneedle-replaceable roller according to claim 1, wherein the support portion comprises:
a clamp arm having an engagement rod adapted to penetratingly pass through a hollow portion formed centrally in the cylindrical rotating body in the direction of a rotational axis of the cylindrical rotating body; and
a fastening member that can be press-fittingly engaged with a distal end of the engagement rod, the fastening member being disposed to opposite to the clamp arm with the roller head interposed between the clamp arm and the fastening member.

4. The microneedle-replaceable roller according to claim 1, wherein the support portion comprises:
a pair of clamp arms each having an end which is free and the other end which is fixed to the handle portion, the width between the clamp arms being gradually increased toward the free ends of the clamp arms; and
a width adjustment ring that surrounds and is moveable near a portion at which the fixed ends of the clamp arms are branched off, such that the width between the clamp arms is adjusted by the movement of the width adjustment ring.

5. The microneedle-replaceable roller according to claim 1, wherein the cylindrical rotating body comprises a retaining groove formed on the outer circumferential surface, the retaining groove having retaining protrusions formed on the inner surface thereof, and
the microneedle sheet comprises sheet openings formed at one end thereof so as to allow the retaining protrusions formed on the retaining groove to be insertedly fitted thereto, and a retaining projection formed at the other end thereof so as to be hookingly retained in the retaining groove.

6. The microneedle-replaceable roller according to claim 5, wherein the support portion comprises:
an engagement rod adapted to penetratingly pass through a hollow portion formed centrally in the cylindrical rotating body in the direction of a rotational axis of the cylindrical rotating body; and
a pair of clamp arms fixedly coupled to both ends of the engagement rod and integrally extending to the handle portion.

7. The microneedle-replaceable roller according to claim 5, wherein the support portion comprises:
a clamp arm having an engagement rod adapted to penetratingly pass through a hollow portion formed centrally in the cylindrical rotating body in the direction of a rotational axis of the cylindrical rotating body; and
a fastening member that can be press-fittingly engaged with a distal end of the engagement rod, the fastening member being disposed to opposite to the clamp arm with the roller head interposed between the clamp arm and the fastening member.

8. The microneedle-replaceable roller according to claim 5, wherein the support portion comprises:
a pair of clamp arms each having an end which is free and the other end which is fixed to the handle portion, the width between the clamp arms being gradually increased toward the free ends of the clamp arms; and
a width adjustment ring that surrounds and is moveable near a portion at which the fixed ends of the clamp arms are branched off, such that the width between the clamp arms is adjusted by the movement of the width adjustment ring.

9. The microneedle-replaceable roller according to claim 1,
wherein the cylindrical rotating body comprises a retaining groove formed on the outer circumferential surface, and
wherein the roller head comprises:
a retaining bar seated in the retaining groove, the retaining bar having retaining protrusions formed on the top surface thereof; and
a pair of side covers joined to both sides of the cylindrical rotating body and securely fixed to both ends of the retaining bar, respectively.

10. The microneedle-replaceable roller according to claim 9, wherein the support portion comprises:
an engagement rod adapted to penetratingly pass through a hollow portion formed centrally in the cylindrical rotating body in the direction of a rotational axis of the cylindrical rotating body; and
a pair of clamp arms fixedly coupled to both ends of the engagement rod and integrally extending to the handle portion.

11. The microneedle-replaceable roller according to claim 9, wherein the support portion comprises:
a clamp arm having an engagement rod adapted to penetratingly pass through a hollow portion formed centrally in the cylindrical rotating body in the direction of a rotational axis of the cylindrical rotating body; and
a fastening member that can be press-fittingly engaged with a distal end of the engagement rod, the fastening member being disposed to opposite to the clamp arm with the roller head interposed between the clamp arm and the fastening member.

12. The microneedle-replaceable roller according to claim 9, wherein the support portion comprises:
a pair of clamp arms each having an end which is free and the other end which is fixed to the handle portion, the width between the clamp arms being gradually increased toward the free ends of the clamp arms; and
a width adjustment ring that surrounds and is moveable near a portion at which the fixed ends of the clamp arms are branched off, such that the width between the clamp arms is adjusted by the movement of the width adjustment ring.

13. A microneedle-replaceable stamp, comprising:
a main body including a handle portion adapted to allow a user to grasp easily; and
a microneedle plate including a plurality of microneedles formed protrudingly downwardly from the bottom surface thereof, the microneedle plate being detachably mounted to the main body.

14. The microneedle-replaceable stamp according to claim 13, wherein the main body comprises a flat microneedle-attaching portion disposed beneath the bottom of the handle portion such that the microneedle plate is detachably mounted on the bottom of the microneedle-attaching portion of the main body.

15. The microneedle-replaceable stamp according to claim 14, wherein the microneedle plate comprises an adhesive layer coated on the top surface thereof.

16. The microneedle-replaceable stamp according to claim 13, wherein the main body comprises an accommodating portion formed vertically protrudingly on the top surface thereof in such a fashion as to extend from the bottom surface thereof,
wherein the microneedle plate comprises a fitting portion formed on the top surface thereof so as to fitted into the accommodating portion, the fitting portion having one or more guide slots formed along the circumference thereof, and
wherein the microneedle-replaceable stamp further comprises a retaining member that is horizontally mounted in the accommodating portion of the main body and is moveable along the guide slots such that the microneedle plate can be securely engaged with the main body.

17. The microneedle-replaceable stamp according to claim 13, wherein the microneedle plate comprises a fitting portion formed on the top surface thereof and having one or more fixing protrusions, and
wherein the main body comprises a recessed portion formed therein so as to allow the fitting portion to be fitted thereto and fixing grooves formed thereon so that when the microneedle plate rotates by a predetermined angle relative to the main body, the fixing protrusions of the fitting portion can be retained in the fixing groove.
